(19) United States
(12) Patent Application Publication
(10) Pub. No.: US 2007/0122504 A1
(43) Pub. Date: May 31, 2007

(54) Grape seed extract having neuronal cell-protection activity and the composition comprising the same for preventing and treating degenerative brain disease

(75) Inventors: Won Kook Moon, SEOUL (KR); Dong Woo Kim, SEOUL (KR); Moo Ho Won, Gangwon-do (KR); In Koo Hwang, SEOUL (KR)

Correspondence Address: KIRK HAHN, 14431 HOLT AVE, SANTA ANA, CA, 92705, US

(73) Assignee: Natural F & P Corp, Seoul (KR)

(21) Appl. No.: 10/595,665

(22) PCT Filed: Nov. 2, 2004
(86) PCT No.: PCT/KR04/02793

§ 371 Date: May 3, 2006

(30) Foreign Application Priority Data
Nov. 7, 2003 (KR) 10-2003-0078053

(51) Int. Cl. A61K 36/87

(52) U.S. Cl. 424/766

(57) ABSTRACT

The present invention relates to a grape seed extract showing neuronal cell protecting activity and the composition comprising the same having neuronal cell protecting activity for preventing and treating degenerative brain disease. The grape seed extract shows potent inhibiting activity of neuronal cell apoptosis occurred in brain ischemia as well as no toxicity, therefore, it can be used as the therapeutics or health food for treating and preventing degenerative brain diseases.

### TECHNICAL FIELD

The present invention relates to a grape seed extract showing neuronal cell protecting activity and the composition comprising the same having neuronal cell protecting activity for preventing and treating degenerative brain disease.

### BACKGROUND ART

In the twentieth century, as the average life span of human has been increasing with the rapid development of life science and medicine, new social problems including increased population ratio of older people are coming to the front, especially, the degenerative neuronal diseases such as stroke, Alzheimer's disease (AD), Parkinson's disease (PD) etc., which are fatal functional disorder of neuronal system, have been increased.

Cerebrovascular disease is classified into two types, hemorrhagic brain disease and ischemic brain disease: hemorrhagic brain disease such as cerebral hemorrhage occurred mainly by some traffic accidents and the ischemic brain disease frequently occurring in older people is caused by the occlusion of cerebral vessels.

In case that temporary cerebral ischemia occurs, the supply of oxygen and glucose to brain is prevented and several syndrome such as ATP decrease and edema follows, which causes to exclusive range of brain injury as the result. After the considerable time lapses, the apoptosis of neuronal cell occurs, which is called as delayed neuronal death. The effect on the delayed type neuronal death is performed by experimenting transient forebrain ischemic model using Mongolian gerbil and it has been reported that neuronal cell death occurs at CA1 region in hippocampus four days after the inducement of cerebral ischemia for 5 mins (Kirino T et al, Acta Neuropathol., 62 pp 201-208, 1984; Kirino T, Brain Res., 239 pp 57-69, 1982).

It has been reported that the neuronal cell death is caused by two mechanisms; one is excitation neuronal cell death mechanism; cerebral ischemia causes to excessive ac cumulation of outer glutamate and those glutamate are influxed into inner cell, which causes to neuronal cell death by excessive accumulation of intracellular calcium ion (Kang T. C. et al, J. Neurocytol., 30, pp 945-955, 2001) and another is oxidation neuronal cell death; the ischemia-reperfusion causing abrupt supply of oxygen resulting in the increase of internal radical ion to give rise to the injury of DNA and cytoplasm (Won M. H. et al, Brain Res., 836 pp 70-78, 1999; Sun A. Y. et al, J. Biomed. Sci., 5, pp 401-141, 998; Flowers F et al, New Horiz 6, pp 169-180, 1998).

On the base of those mechanism study, there have been endeavored to develop effectively inhibiting substance of neuronal cell death or the mechanism thereof till now, however, the effective and satisfactory inhibitors of neuronal cell death have been not found yet.

t-PA (tissue plasminogen activator), sole approved cerebral ischemia treating agent in FDA and sold in the market is a thrombolytic agent dissolving thrombus causing cerebral ischemia and inducing rapid supply of oxygen and glucose. Accordingly, it could not protect neuronal cell directly, therefore it should be used urgently. Furthermore, since it is thrombolytic agent, hemorrhagic cerebral disease occurs in case that it is administrated in over dose or too frequently.

MK-801, a potent calcium channel blocker effectively inhibiting initial calcium ion influx had been on clinical trial however it has been abandoned because of its adverse action.

In South Korea, lots of health care food containing natural substance have been on the market however most of those are not yet authorized by scientific test and abused to give rise to scientific problems in the end.

Accordingly, there have been still needed to develop novel natural resource effective in treating and preventing cerebral disease through substantive and scientific experiments till now.

It have been reported that grape seed extract contains high amount of catechin showing various activities such as intoxifying, bacteriocidal and anticancer activities and picnagerol showing strengthening blood vessel and shows antioxidant effect to inhibit the action of harmful oxygen. (Shi J. et al, J. Med. Food 6, pp 291-299, 2003).

However, there has been not reported or disclosed about therapeutic effect for brain disease of grape seed extract MA in any of above cited literatures, the disclosures of which are incorporated herein by reference.

To investigate an effect of grape seed extract on the neuronal cell death through several biochemical experiments the inventors of the present invention have intensively carried out several experiments such as in vitro test determining inhibiting effect on LDH enzyme and in vivo animal test using Mongolian gerbil and finally completed present invention by confirming that the grape seed extract inhibits the neuronal cell death and show neuronal cell protective activity .

These and other objects of the present invention will become apparent from the detailed disclosure of the present invention provided hereinafter.

### DISCLOSURE

The present invention provides a pharmaceutical composition comprising a grape seed extract as an active ingredient in an effective amount to treat and prevent degenerative brain disease by protecting neuronal cell.

The present invention also provides a use of grape seed extract for the manufacture of the medicament to treat and prevent degenerative brain disease by protecting neuronal cell in mammal or human in need thereof.

The present invention also provides a health food or food additives comprising grape seed extract for the prevention or alleviation of degenerative brain disease by protecting neuronal cell.

Accordingly, it is an object of the present invention to provide a purified grape seed extract prepared by the steps consisting of: extracting grape seed with alkaline distilled water to obtain alkaline soluble substance; neutralizing with acidic solution, centrifuging to obtain precipitated layer; suspending the precipitate with alcohol, centrifuging to obtain supernatant layer; concentrating the supernatant, adding non-polar solvent, removing non-polar solvent soluble layer to obtain purified fraction; subjecting to repeated purification and lyophilzation to obtain dried grape seed extract of the present invention showing potent neuronal cell protective activity.

It is an object of the present invention to provide a pharmaceutical composition comprising grape seed extract as an active ingredient for the treatment and prevention of brain disease by protecting neuronal cell.

The term ‘extract’ disclosed herein means all the extract prepared by extracting with water, lower alcohols such as methanol, ethanol, preferably methanol and the like, or the mixtures thereof, preferably water, more preferably the extract prepared by above described steps.

It is an object of the present invention to provide a use of grape seed extract for the manufacture of the medicament to treat and prevent degenerative brain disease by protecting neuronal cell in mammal or human in need thereof.

It is an object of the present invention to provide a method of treating or preventing degenerative brain disease by protecting neuronal cell in a mammal comprising administering to said mammal an effective amount of grape seed extract, together with a pharmaceutically acceptable carrier thereof.

It is another object of the present invention to provide a health food or food additives comprising grape seed extract, together with a sitologically acceptable additive for the prevention and improvement of degenerative brain disease by protecting neuronal cell.

Above described degenerative brain disease comprises stroke, cerebral concussion, Huntington's disease, Creutzfeld-Jakob disease, Alzheimer's disease (AD), Parkinson's disease (PD), senile dementia and the like.

The pharmaceutical composition of the present invention can contain about 0.01˜50% by weight of the above extract based on the total weight of the composition.

The health food of the present invention comprises above extracts as 0.01 to 80%, preferably 1 to 50% by weight based on the total weight of the composition.

Above health food can be contained in health food, health beverage etc, and may be used as powder, granule, tablet, chewing tablet, capsule, beverage etc.

An inventive extracts isolated from grape seed may be prepared in accordance with the following preferred embodiment.

Hereinafter, the present invention is described in detail.

The inventive grape seed extract can be prepared by follows;

At 1^{st} step, grape seed is dried, crushed and mixed with 5 to 20-fold, preferably, approximately 8 to 12 fold volume of distilled water, lower alcohols such as methanol, ethanol, butanol and the like, or the mixtures thereof, preferably water; the seed is extracted with alkaline distilled water adjusted to the pH ranging from 8 to 11, preferably, at the temperature ranging from 20 to 50° C., preferably at R. T., for the period ranging from 1 to 36 hours, preferably 12 to 24 hours with conventional extraction method by stirring extraction with water, reflux extraction, or ultra-sonication extraction, preferably stirring extraction method, with 1 to 5 times, preferably 2 to 3 times, to obtain alkaline soluble substance;

In the above step, if the pH of alkaline solution is less than 8.0, the extraction efficiency is decreased significantly therefore it is preferable to adjust the pH of solution to more than 8.0.

At 2^{nd} step, neutralizing with acidic solution such as strong acid for example, hydrochloric acid to adjust to the pH ranging from 2 to 4, preferably 3 and centrifuging to obtain precipitated layer are preferable;

At 3^{rd} step, adding about 3 to 7 fold weight of lower alcohol such as methanol or ethanol, suspending, centrifuging to obtain supernatant layer and consequently concentrating the supernatant to obtain its concentrates are preferable;

At 4^{th} step, adding non-polar solvent, removing non-polar solvent soluble layer to obtain purified fraction and subjecting to repeated purification and lyophilzation to obtain dried grape seed extract of the present invention showing potent neuronal cell protective activity are preferable.

Accordingly, the present invention also provide above described method for preparing inventive grape extract of the present invention showing potent neuronal cell protective activity.

The present invention provides a purified grape extract prepared by above-described step showing potent neuronal cell protective activity.

The term ‘grape seed extract’ disclosed herein refers to all the seed extract extracted from Vitis genus such as Vistis vinifera L, Vitis vinifera, Vitis labrusca, Vitis riparia, Vitis rupestris, Vitis berladieri, Vitis coignetiae Pulliat ex Planchon, Vitis amurensis Ruprecht, Vitis ficifolia Bunge, Vitis flexuosa Thunb and so on.

Present inventors have performed several experiments such as in vitro test determining inhibiting effect on LDH enzyme in PC12 cell line and in vivo animal test using Mongolian gerbil and finally confirmed that the grape seed extract inhibit the neuronal cell death and show potent preventing activity of the neuronal cell injury caused by brain ischemia.

In accordance with another aspect of the present invention, there is provided a pharmaceutical composition comprising grape seed extract prepared by above preparation method for the treatment and prevention of degenerative brain disease by protecting neuronal cellas active ingredients.

It is another of the present invention to provide a treating method and preventing method comprising administering a pharmaceutical composition comprising said extract prepared by above preparation method to degenerative brain of mammals including human.

The inventive composition for treating and preventing degenerative brain disease by protecting neuronal cell may comprises above extracts as 0.001˜50% by weight based on the total weight of the composition. If the amount of seed extract is less than 0.001% (w/w), over dosing administration may be required to obtain effective efficacy and if the amount of seed extract is more than 50% (w/w), it is not economical since the efficacy of abundant extract may be equal to that of lesser amount of extract.

However, it is preferable that the amount of extract is controlled according to the using method and the using purpose of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-aglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol, plasters and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging from 50 mg/kg to 500 mg/kg per day, by weight/day of the inventive extract or compounds of the present invention. The dose may be administered in single or divided into several times per day. In terms of composition, the amount of inventive extract should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Also, the present invention provide a composition of the health care food for the prevention and improvement of degenerative brain disease by protecting neuronal cell adding above described extracts 0.01 to 80% by weight, amino acids 0.001 to 5% by weight, vitamins 0.001 to 2% by weight, sugars 0.001 to 20% by weight, organic acids 0.001 to 10% by weight, sweetener and flavors of proper amount in the form of beverage.

Above described seed extract can be added to food and beverage for the prevention and improvement of degenerative brain disease by protecting neuronal cell.

To develop for health food, examples of addable food comprising above extracts of the present invention are various food, beverage, gum, vitamin complex, health improving food and the like, and can be used as power, granule, tablet, chewing tablet, capsule or beverage etc.

Above described composition therein can be added to food, additive or beverage, wherein, the amount of above described extract in food or beverage may generally range from about 0.1 to 80 w/w %, preferably 1 to 50 w/w % of total weight of food for the health food composition and 1 to 30 g, preferably 3 to 10 g on the ratio of 100 ml of the health beverage composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sir such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, alpha-tocopherol, rosemary extract, vitamin C, green tea extract, licorice root extract, chitosan, tannic acid, phytic acid etc.

The above grape seed extract may be 20 to 90% high concentrated liquid, power, or granule type.

Similarly, the above seed extract can comprise additionally one or more than one of lactose, casein, dextrose, glucose, sucrose and sorbitol.

Inventive extract of the present invention have no toxicity and adverse effect therefore; they can be used with safe.

It will be apparent to those skilled in the art that various modifications and variations can be made in the compositions, use and preparations of the present invention without departing from the spirit or scope of the invention.

### DESCRIPTION OF DRAWINGS

The above and other objects, features and other advantages of the present invention will more clearly understood from the following detailed description taken in conjunction with the accompanying drawings, in which;

FIG. 1 shows the preventive effect of grape seed extract administrated into PC12 cell line on the neuronal cell death in the low oxygen environment determined by LDH determination experiment;

FIG. 2 shows stained photographs of the hippocampus region in the brain tissue of experimental animal C and C are normal groups four days after the ischemia-reperfusion treatment, B and D are control groups treated with only solvent, C and D are magnified photographs of A and B in the region of CA1 (400×));

FIG. 3 shows stained photographs of the hippocampus region in the brain tissue of experimental animal four days after the ischemia-reperfusion treatment (A and C are photographs of 30 mins before the treatment and B and D are photographs of 30 mins after the treatment);

FIG. 4 represents magnified photograph of CA1 region in Hippocampus tissue depicted in FIG. 3 (400×);

FIG. 5 represents the comparison of the number of viable cells between control group and test groups four days after the treatment with grape seed extract to the experimental animals 30 mins before and after the ischemia-reperfusion treatment.

### MODE FOR INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Example 1

### Preparation of Inventive Extract of Grape Seed

### 1-1. Water Extract of Grape Seed

1 kg of dried and crushed grape seed (Vistis vinifera L), purchased from Kyungdong Market located in Seoul was mixed with 10 folds volume of distilled water and stirred. Appropriate amount of NaOH was added thereto to adjust the pH to 10 and stirred to extraction for 6 hours at R. T. The obtained extract was acidified with HCl to adjust the pH to 3.0, subjected to centrifugation to collect 100 g of precipitates. 5 folds of ethanol (w/w) was added to the concentrates, suspended and subjected to centrifugation to obtain the supernatant. The supernatant was concentrated to obtain 50 g of concentrates and equal amount of hexane was added thereto to remove hexane soluble layer. Remaining lower layer was dried with lyopholization to obtain 30 g of brown dried powder.

### 1-2. Ethanol Extract of Grape Seed

1 kg of dried and crushed grape seed (Vitis vinifera) purchased from Kyungdong Market located in Seoul was mixed with 10 folds volume of 100% ethanol and extracted with stirring for 12 hours at R. T. The extract was filtered and concentrated in vacuo to be 1/10 volume of extract. 50 g of concentrates was mixed with equal amount of hexane and fractionated with repeated extraction to remove hexane soluble layer. Remaining lower layer was concentrated and dried with lyopholization to obtain 30 g of brown dried powder.

### 1-3. Sample Preparation

The dried grape seed extracts prepared from above Example 1-1 and 1-2 were dissolved with 10 ml of distilled water with a ratio of 100 mg of extract/ml of distilled water to use as a test sample in following Experiments.

### Experimental Example 1

### LDH Release in Vitro Assay of Grape Seed Extract

The LDH (lactate dehydrogenase) inhibitory effect of inventive extract was investigated to measure the extent of apoptosis by them.

PC12 cell was treated with COCl₂ to induce low oxygen environment to cause the injury of neuronal cell. To determine whether the injury of neuronal cell occur or not, the LDH concentration released from extra-cellular medium in culture cell was determined. The cell medium was collected at 20 to 24 hours after the treatment, i.e., at the time of completing the release of enzyme, and the concentration of released enzyme was determined using by microplate reader.

The PC 12 cell was treated with various concentrations of grape seed extract, i.e., 0, 10, 50, 100, 500 and 500 ?g/ml, before and after the inducement of low oxygen environment and cultivated for 20 to 24 hours at 37° C. to obtain cell culture medium. The concentration of LDH was determined using by Beckman DU-640 absorption spectrophotometer according to enzyme dynamic method using by Zhong Sheng Biotech standard reagent.

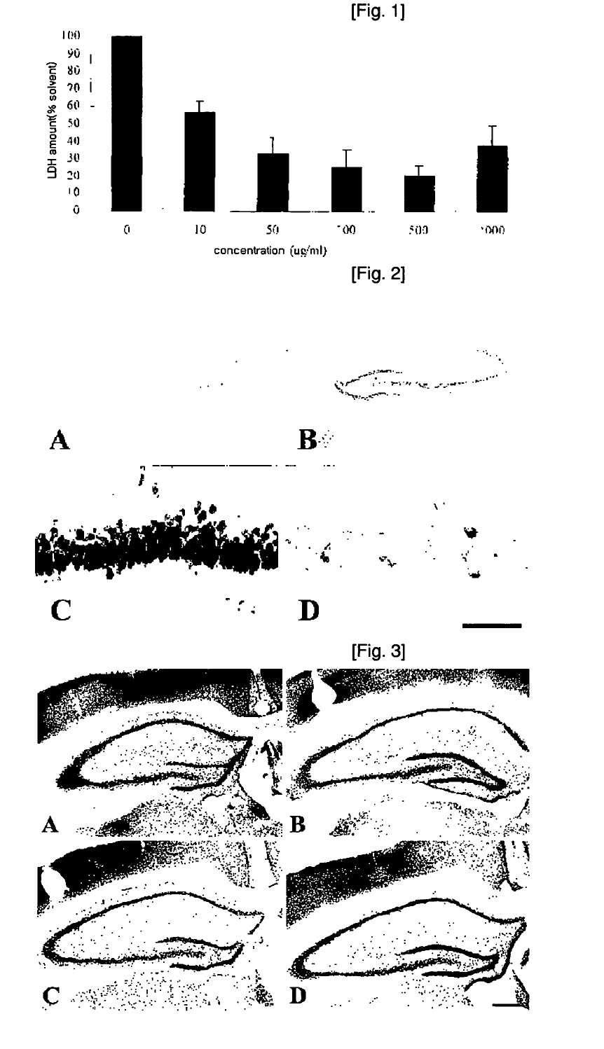

The result was shown in FIG. 1. The test groups treated with grape seed extract before and after the inducement of low oxygen environment reduced the release of LDH in dose dependant manner compared with those of control group treated with only solvent. Accordingly, it is confirmed that the grape seed extract reduced apoptosis induced by low oxygen environment significantly.

### Experimental Example 2

### Effect of Grape Seed Extract on Neuronal Cell Using by in Vivo Animal Experiment

To determine the effect of grape seed extract on apoptosis of neuronal cell, following animal experiment was performed in this experiment.

### 2-1. The Breeding of Experimental Animal and Administration Method

60 numbers of male and female Mongolian gerbil (Meriones unguiculatus) weighing 65 to 75 g were used as experimental animals.

The animals providing with free access to water and feed were acclimated with following breeding condition maintaining the temperature of 23±2° C. and the relative humidity of 55±10 ° C. under the regularly controlled light/dark condition, i.e., light from am 7:00 to pm 7:00.

### 2-2. Experimental Procedure

0.5 ml of the grape seed extract prepared in Example 1-3 was orally administrated into experimental animals 30 minutes before and after the inducement of ischemia as a test group and the non-treated group was used as control group. The experimental animals were orally anesthetized with 5% isoflurane gas (the gas mixture consisting of 70% N₂O and 30% O₂ gas and 5% isoflurane) and maintained with 2.5% isoflurane gas during the experiment.

The disinfected skin at the center of mouse neck was excised and right-handed CCA (common carotid artery) and ECA (external carotid artery) were isolated from neighboring tissue and nerves with care. CCA and ECA were ligated for 5 minutes with aneurysm clip (Staelting, USA ) to induce ischemia and the clips were removed to provide with repurfusion. Similar surgery to test group was performed as a control group. The complete occlusion of CCA in test groups was confirmed by observing the blood circulation of central artery of retina with ophthalmoscope.

The body temperature of animals during the inducement of ischemia was determined by inserting thermometer into rectum and was maintained at 37±0.3 ° C. using by automatically controlled heat pad according to the experimental temperature.

4 days after the ischemia inducement, each 40 mg/kg amount of thiopental sodium (Yuhan Co. Korea) was intraperitoneally administrated to normal group, control group and test group respectively to anesthetize and physiological saline solution containing 1000 IU heparin per 1000 ml solution maintaining the temperature of 4° C. was injected into left ventricle to perfusive washing. Perfusion fixation was performed using 4% paraformaldehyde containing 0.1M phosphate buffer (pH 7.4) and the brain of animal was delivered and fixed with fixation solution for 4 to 6 hour. The fixed brain was dipped in 30% sucrose solution in 0.1 M phosphate buffer. The brain was sliced into 20 ?m width of tissue slices using by sliding microtome (Reichert-Jung Co. Germany) and the slices were stored at 6 well plate containing storing solution at 4° C. before use.

The tissue wherein hippocampus formation was well developed was selected among the all slices and washed with 0.01M PBS three times for 10 minutes to remove remaining storing solution. The slice was transferred to the gelatin coated slide and dried at 37° C. sufficiently, dipped in distilled water for a while and stained with 2% cresyl violet acetate (Signa Co. USA). The tissue was washed with running tap water to remove remaining staining reagent, dipped in distilled water for a while, treated with 50%, 70%, 80%, 90%, 95% and 100% solution, dehydrated and remaining staining agent was removed by washing. After confirming the detection of Nissle body in the tissue, the tissue was dipped in xylen reagent (Junsei Co. Japan) to be transparent and sealed with Canadian Balsam (Kanto Co. Japan).

The CA1 region of each tissue was photographed by Axioplan microscope equipped with digital camera (Carl Zeiss Co. Germany) magnified with 1000×. The violet stained region was selected using by image analyzer (Optimas 6.5, USA ) program and the number of neuronal cells was counted. To verify the significance for each group, the number of live neuronal cell was divided into that of normal cell and the result was expressed with percentage (%). One way ANOVA test was performed for statistical analysis and most general region selected from each group was photographed by Axioplan microscope equipped with digital camera (Carl Zeiss Co. Germany).

### 2-3. Experimental Result

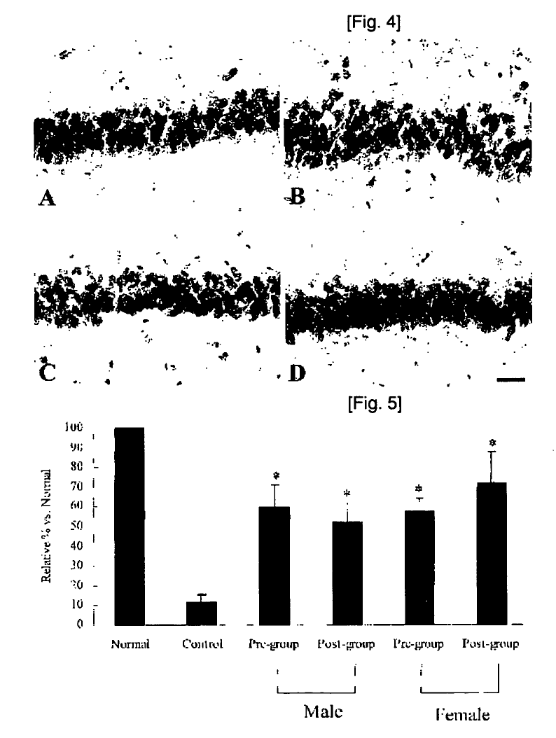

As can be seen in FIG. 2, neuronal cell was observed in normal group while not detected in control group since all the neuronal cells were died from ischemia In FIGS. 3, A and B are the photographs treated with grape seed extract 30 minutes before ischemia inducement whereas C and D are the photographs treated with grape seed extract 30 minutes after ischemia inducement A and B are photographs of male while C and D are of female. As can be seen in FIG. 3, CA 1 region of tissues was stained with dark color, which means that all the test groups treated with grape seed extract show potent inhibiting activity of apoptosis. The magnified photograph with 400×, i.e., FIG. 4, shows that the cell shapes of test group and negative control groups are similar to each other therefore, it is confirmed that the grape seed extract has potent inhibiting activity of neuronal cell death.

FIG. 5 shows the determined result of the experiment confirming the effect of grape seed on the apoptosis of neuronal cell after ishemia-reperfusion treatment The determined number of living neuronal cells for each group, i.e., normal group Normal), control group (Control), test group treated with grape seed extract before ischemia-repurfusion treatment (pre-male and pre-female), test group treated with grape seed extract after ischemia-repurfusion treatment (post-male and post-female) was divided into that of negative control group and the calculated value was expressed as percentage. Asterisk (*) denotes the effective group with 99% of statiscal significance level.

As can be shown in FIG. 5, the survival ratio of control group showed higher rate (about 11.6%) than that of normal group however, the survival ratio of test group showed highest rate among the groups. The survival ratio of neuronal cell in grape seed treated group to male and female animals showed 59.6 and 57.4% in treatment group before the inducement of brain ischemia, whereas 51.9 and 71.9% in treatment group after the inducement of brain ischemia, respectively.

Considering the fact that the survival rate of neuronal cell treated with positive control group (Ebselen) ranges from 50 to 60%, it is confirmed that the grape seed extract of the present invention can be useful in treating and preventing degenerative brain disease such as ischemia, stroke and so on as a medicine, health care food in spite of natural resource.

### Experimental Example 3

### Toxicity Test

### Methods (1)

The acute toxicity tests on ICR mice (mean body weight 25±5 g) and Spree-Dawley rats (235±10 g, Jung-Ang Lab Animal Inc.) were performed using the extract of the Example 1-1. Four group consisting of 10 mice or rats was administrated orally intraperitoneally with 250 mg/kg, 500 mg/kg, 1000 mg/kg and 5000 mg/kg of test sample or solvents (0.2 ml, i.p.) respectively and observed for 2 weeks.

### Methods (2)

The acute toxicity tests on ICR mice and Sprague-Dawley rats were performed using the extract of the Example 1-2. Four group consisting of 10 mice or rats was administrated intraperitoneally with 25 mg/kg, 250 mg/kg, 500 mg/kg and 725 mg/kg of test sample or solvents (0.2 ml, i.p.), respectively and observed for 24 hours.

### Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of powder

| | |
|---|---|
| Dried powder of Example 1-1 | 50 mg |
| Lactose | 100 mg |
| Talc | 10 mg |

Powder preparation was prepared by mixing above components and filling sealed package.

Preparation of tablet

| | |
|---|---|
| Dried powder of Example 1-1 | 50 mg |
| Corn Starch | 100 mg |
| Lactose | 100 mg |
| Magnesium Stearate | 2 mg |

Tablet preparation was prepared by mixing above components and entabletting.

Preparation of capsule

| | |
|---|---|
| Dried powder of Example 1-1 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of injection

| | |
|---|---|
| Dried powder of Example 1-1 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of liquid

| Dried powder of Example 1-2 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of health food

| | |
|---|---|
| Extract of Example 1-2 | 1000 mg |
| Vitamin mixture | optimum amount |
| ?Vitamin A acetate | 70 ?g |
| ?Vitamin E | 1.0 mg |
| ?Vitamin B₁ | 0.13 mg |
| ?Vitamin B₂ | 0.15 mg |
| ?Vitamin B₆ | 0.5 mg |
| ?Vitamin B₁₂ | 0.2 ?g |
| ?Vitamin C | 10 mg |
| ?Biotin | 10?g |
| ?Amide nicotinic acid | 1.7 mg |
| ?Folic acid | 50 ?g |
| ?Calcium pantothenic acid | 0.5 mg |
| Mineral mixture | optimum amount |
| ?Ferrous sulfate | 1.75 mg |
| ?Zinc oxide | 0.82 mg |
| ?Magnesium carbonate | 25.3 mg |
| ?Monopotassium phosphate | 15 mg |
| Dicalcium phosphate | 55 mg |
| Potassium citrate | 90 mg |
| Calcium carbonate | 100 mg |
| Magnesium chloride | 24.8 mg |

The above-mentioned vitamin and mineral mixture may be varied in may ways. Such variations are not to be regarded as a departure from the spirit and scope of the present invention.

Preparation of health beverage

| | |
|---|---|
| Extract of Example 1-2 | 1000 mg |
| Citric acid | 1000 mg |
| Oligosaccharide | 100 g |
| Apricot concentration | 2 g |
| Taurine | 1 g |
| Distilled water | 900 ml |

Health beverage preparation was prepared by dissolving active component, mixing, stirring at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the grape seed extract shows potent inhibiting activity of neuronal cell apoptosis occurred in brain ischemia as well as no toxicity, therefore, it can be used as the therapeutics or health food for treating and preventing neuro-degenerative brain diseases.

1. A method to prepare purified grape seed extract comprising the steps of:
a. extracting grape seed with alkaline distilled water to obtain alkaline soluble substance;
b. neutralizing with acidic solution,
c. centrifuging to obtain precipitated layer;
d. suspending the precipitate with alcohol,
e. centrifuging to obtain supernatant layer;
f. concentrating the supernatant,
g. adding non-polar solvent,
h. removing non-polar solvent soluble layer to obtain purified fraction;
i. subjecting to repeated purification and,
j. lyophilzing to obtain dried grape seed extract of the present invention showing potent neuronal cell protective activity.

2. A pharmaceutical composition comprising grape seed extract as an active ingredient for the treatment and prevention of degenerative brain disease.

3. The pharmaceutical composition of claim 2 wherein said grape seed extract is extracted with a solvent selected from the group consisting of water, lower alcohol and mixtures thereof.

4. The pharmaceutical composition of claim 2 wherein said grape seed extract is prepared by the method of claim 1.

5. The pharmaceutical composition of claim 2 wherein said degenerative brain disease selected from the group consisting of stroke, cerebral concussion, Huntington's disease, Creutzfeld-Jakob disease, Alzheimer's disease (AD), Parkinson's disease (PD), and senile dementia.

6. A health food comprising grape seed extract mixed with a sitologically acceptable additive for the prevention and improvement of degenerative brain disease.

7. The pharmaceutical composition of claim 2 wherein said grape seed extract is extracted with water.

8. The pharmaceutical composition of claim 7 wherein the water is alkaline.

9. The health food of claim 6, wherein the grape seed extract comprises between 0.01% and 80% of total weight.

10. The health food of claim 9, wherein the grape seed extract comprises between 1% and 50% of total weight.

11. The pharmaceutical composition of claim 5 wherein said degenerative brain disease is stroke.

12. The pharmaceutical composition of claim 5 wherein said degenerative brain disease is Alzheimer's disease.

13. The pharmaceutical composition of claim 5 wherein said degenerative brain disease is senile dementia.
